(19) United States
(12) Patent Application Publication
(10) Pub. No.: US 2015/0174079 A1
(43) Pub. Date: Jun. 25, 2015

(54) LOZENGES WITH SILVER NANOPARTICLES

(71) Applicant: Food for Health International, LLC, Orem, UT (US)

(72) Inventors: Frank Davis, Orem, UT (US); Andrew Atkinson, American Fork, UT (US); Elaine Atkinson, American Fork, UT (US)

(21) Appl. No.: 14/582,662

(22) Filed: Dec. 24, 2014

Related U.S. Application Data
(60) Provisional application No. 61/920,606, filed on Dec. 24, 2013.

(51) Int. Cl. A61K 9/51; A61K 9/00; A61K 33/38

(52) U.S. Cl. CPC A61K 9/5192; A61K 33/38; A61K 9/5176; A61K 9/0056

(57) ABSTRACT

A solid lozenge composition for oral dissolution. The solid lozenge composition includes an inactive carrier medium that is a solid at room temperature, at least one flavoring component, and silver nanoparticles. The concentration of silver nanoparticles is between about 0.1 micrograms per teaspoon and 5 micrograms per teaspoon.

### CROSS-REFERENCES TO RELATED APPLICATIONS

This application claims the benefit of U.S. Provisional Patent Application No. 61/920,606 filed on Dec. 24, 2013 for Frank Davis, which is incorporated herein by reference.

### FIELD

This invention relates to lozenges and more particularly relates to lozenges with silver nanoparticles.

### BACKGROUND

Silver has been employed as a germicide and antibiotic for a number of years and has been used in the past by civilizations before modern antibiotics were developed. For example, in previous centuries users would shave silver particles into their drinking water, or submerge whole silver pieces in the drinking water, for the purpose of ingesting the silver by drinking.

More recently, silver has been implemented in liquid mixtures and have been made available for ingestion. Many conventional silver-based products, however, are unstable and the effectiveness of the silver is at least diminished due to precipitation, aggregation, or settling (i.e., silver ‘falls out” of solution). In other words, many conventional silver products fail to maintain the silver particles in suspension, either because the silver solution is not a true colloid or because it is otherwise unstable. When the suspension of the silver particles fails, the particles fall to the bottom of the solution, thereby reducing the solution's concentration of silver (or at least decreasing the uniformity of the concentration, thus making spatial concentrations relatively inconsistent) and rendering the silver product less effective.

### SUMMARY

The subject matter of the present disclosure has been developed in response to the present state of the art of throat lozenges. Accordingly, the subject matter of the present disclosure has been developed to provide a solid lozenge composition that overcomes many shortcomings in the prior art.

Disclosed herein is one embodiment of a solid lozenge composition for oral dissolution. The solid lozenge composition includes an inactive carrier medium that is a solid at room temperature, at least one flavoring component, and silver nanoparticles. The concentration of silver nanoparticles is between about 0.1 ppm and 25 ppm.

In one implementation, the solid lozenge composition further includes a silver dispersing medium that, independent from the inactive carrier medium, is a liquid at room temperature. In another implementation, the solubility of the solid lozenge composition in a mouth of a user and the size of an individual lozenge are such that it takes at least 6 minutes to completely dissolve each individual lozenge. Still further, the inactive carrier medium may make up more than 50 volume percent of the solid lozenge composition.

In one implementation, the silver nanoparticles are produced via electrolysis. In another implementation, the inactive carrier medium is evaporated cane juice. Also, the solid lozenge composition may include a consistency modifier and in certain implementations the consistency modifier may be honey. In one implementation, the flavoring component is an essential oil.

In another implementation, the concentration of silver nanoparticles is between about 2 ppm and 10 ppm. In yet another implementation, the concentration of silver nanoparticles is about 4.25 ppm. According to another implementation, the concentration of silver nanoparticles is about 20 micrograms per 4.5 gram lozenge.

Also disclosed herein, according to one embodiment, is a method for using solid lozenges containing silver nanoparticles. The method includes providing a solid lozenge composition for oral dissolution. The solid lozenge composition comprising an inactive carrier medium that is a solid at room temperature, at least one flavoring component, and silver nanoparticles, wherein the concentration of silver nanoparticles is between about 0.1 and 25 ppm. The method further includes placing the solid lozenge composition in a mouth of a user, and then orally dissolving the solid lozenge composition for a time period of at least 2 minutes, during which time period the silver nanoparticles are in substantially constant and repeated contact with a discomfort source in the throat of the user. According to one implementation, the time period is at least 6 minutes.

Further disclosed herein is a method, according to one embodiment, for making solid lozenges that contain silver nanoparticles. The method includes combining an inactive carrier medium with silver nanoparticles over low heat to produce a liquid mixture, with the concentration of silver nanoparticles in the mixture being between about 0.1 and 25 ppm. The method further includes heating the liquid mixture to a first temperature, stifling the liquid mixture frequently and heating the liquid mixture to a second temperature, removing the liquid mixture from heat and continue stifling until the temperature of the liquid mixture falls to a third temperature, and then adding at least one essential oils flavoring component and continue stifling until the liquid mixture looks smooth and congruous. Finally, the method includes dispensing the liquid mixture into lozenge molds and allowing the liquid mixture to cool into a solid.

According to one implementation, the method further includes initially combining a consistency modifier with the inactive carrier medium and the silver nanoparticles to produce the liquid mixture. In another implementation, the second temperature is higher than the first temperature and the third temperature is between the first and the second temperatures. For example, the first temperature may be 250 degrees Fahrenheit, the second temperature may be 295 degrees Fahrenheit, and the third temperature may be 280 degrees Fahrenheit.

Reference throughout this specification to features, advantages, or similar language does not imply that all of the features and advantages that may be realized with the present disclosure should be or are in any single embodiment of the invention. Rather, language referring to the features and advantages is understood to mean that a specific feature, advantage, or characteristic described in connection with an embodiment is included in at least one embodiment of the subject matter disclosed herein. Thus, discussion of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize that the subject matter of the present application may be practiced without one or more of the specific features or advantages of a particular embodiment. In other instances, additional features and advantages may be recognized in certain embodiments that may not be present in all embodiments of the disclosure. Further, in some instances, well-known structures, materials, or operations are not shown or described in detail to avoid obscuring aspects of the subject matter of the present disclosure. These features and advantages of the present disclosure will become more fully apparent from the following description and appended claims, or may be learned by the practice of the disclosure as set forth hereinafter.

### BRIEF DESCRIPTION OF THE DRAWINGS

In order that the advantages of the subject matter of the present disclosure will be readily understood, a more particular description of the subject matter will be rendered by reference to specific embodiments that are illustrated in the appended drawings. Understanding that these drawings depict only typical embodiments of the subject matter of the present disclosure and are not therefore to be considered to be limiting of its scope, the subject matter will be described and explained with additional specificity and detail through the use of the accompanying drawings, in which:

FIG. 1A depicts multiple lozenges in a lozenge container, according to one embodiment;

FIG. 1B depicts a lozenge wrapped in a wrapper, according to one embodiment;

FIG. 2A is a schematic block diagram of a solid lozenge composition, according to one embodiment;

FIG. 2B is a schematic block diagram of the solid lozenge composition, according to another embodiment;

FIG. 3A depicts a user orally taking a solid lozenge, according to one embodiment;

FIG. 3B depicts a user orally taking a solid lozenge, according to another embodiment;

FIG. 4 is a schematic flow chart diagram of a method for making a solid lozenge, according to one embodiment; and

FIG. 5 is a schematic flow chart diagram of a method for using a solid lozenge composition, according to one embodiment.

### DETAILED DESCRIPTION

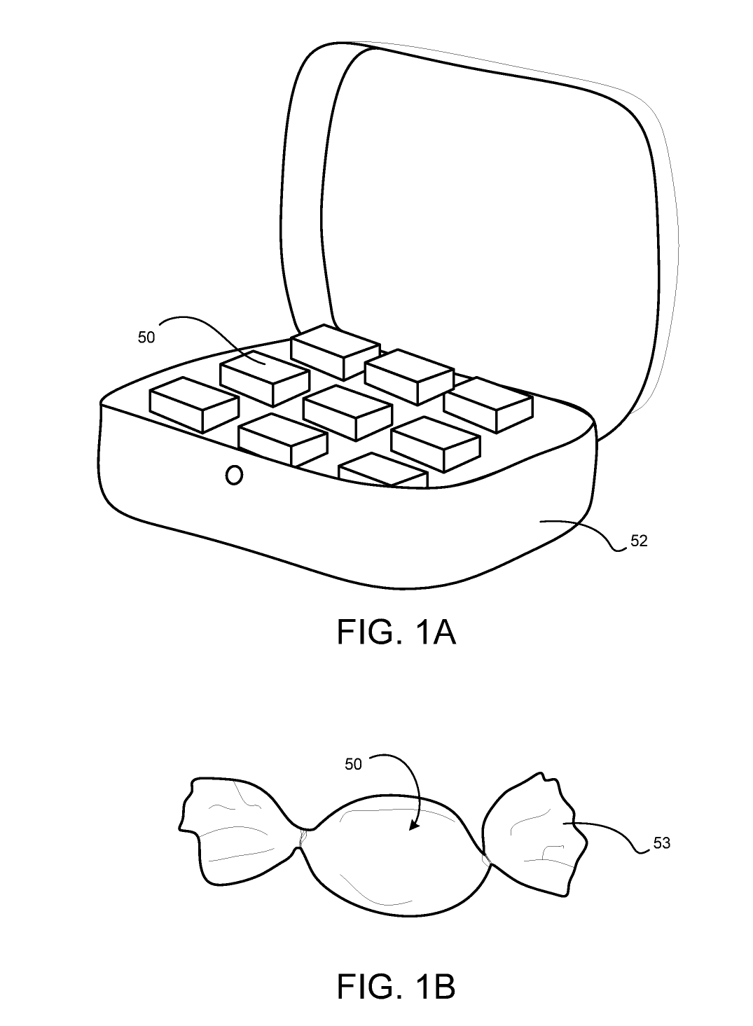

FIG. 1A depicts multiple lozenges 50 in a lozenge container 52, according to one embodiment. Lozenges 50, often referred to as cough-drops, are small tablets that are designed to be dissolved in the mouth. As a lozenge dissolves in the mouth of a user, the medication and/or active ingredient(s) within the lozenge lubricates and soothes the throat, suppresses coughs, kills bacteria, and/or generally improves the health and comfort of the user. Lozenges 50 may contain a variety of constituents, which are described below in greater detail.

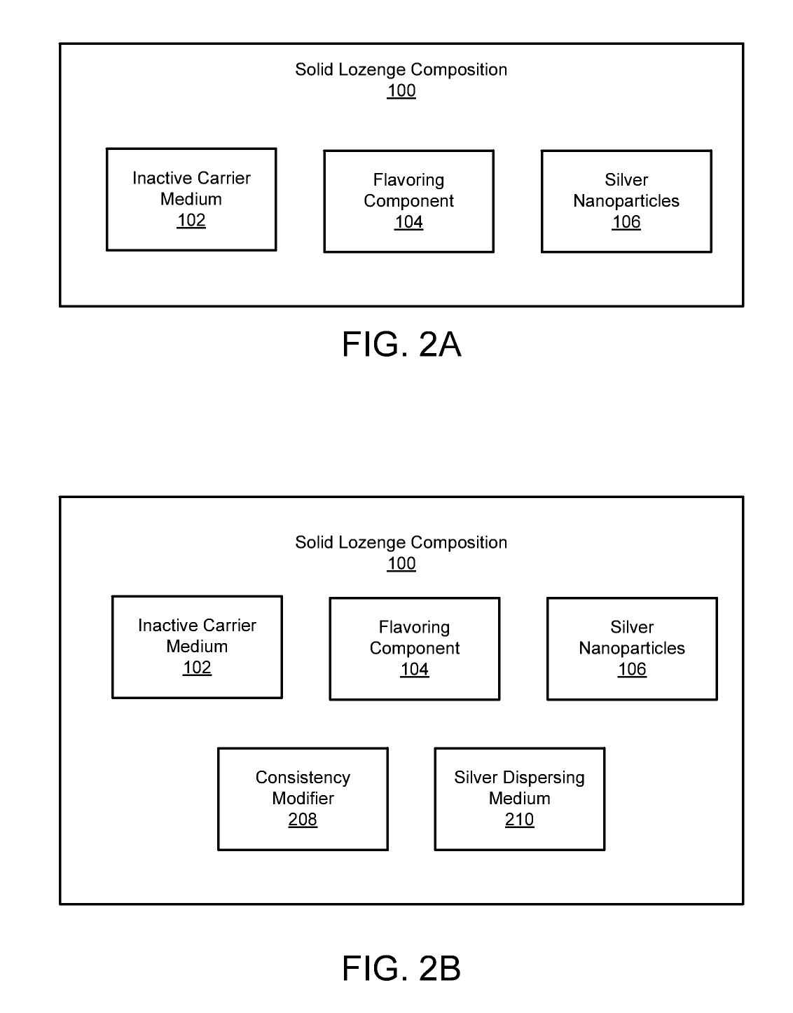

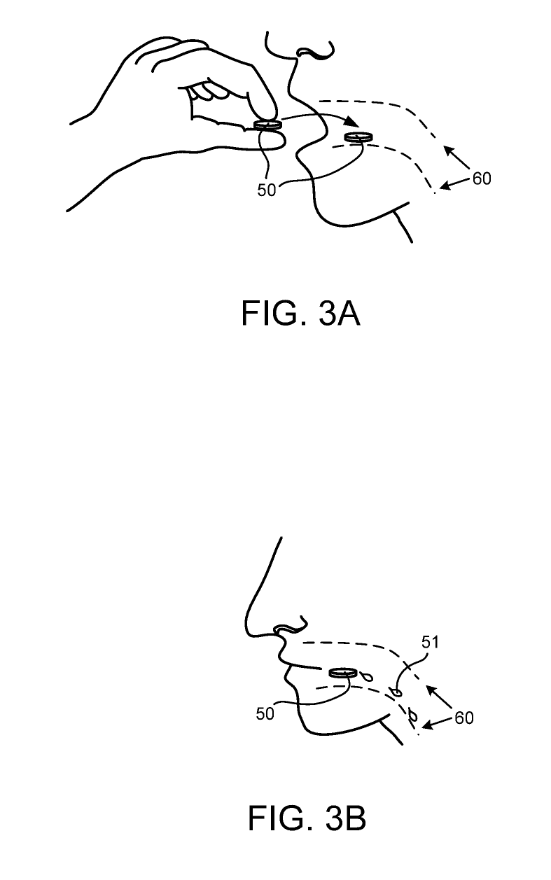

As depicted in FIG. 1A, lozenges 50 may have a specific shape and may be packaged together in specific lozenge containers 52. For example, lozenges 50 may have a rectangular shape and may be sized so that an adult user can easily place a lozenge in his mouth. In another embodiment, not depicted, the lozenges 50 may be shaped like an oval or a conventional cough-drop. In yet another embodiment, the lozenges 50 may be attached to the end of stick in the form of lollipop. In one embodiment, the lozenges 50 are packaged in containers 53 that hold multiple individual lozenges 50. The lozenges 50 within the containers 53 may be separated by partitions within the container 53 or may have wax paper or other similar material separating the lozenges to prevent them from sticking together. The lozenges 50 may also be coated with a powdered covering, such as evaporated powdered cane juice. In one embodiment, the covering may have a certain flavor or may have a certain taste. In another embodiment, the covering may prolong the useful life of the lozenges. Additional details regarding lozenges 50, their composition, and their method of manufacture are included below with reference to FIGS. 2A-2B, 3A, -3B, and 4.

FIG. 1B depicts a lozenge 50 wrapped in a wrapper 53, according to one embodiment. According to the depicted embodiment, lozenges 50 may also be individually packaged in wrappers 53. The individually wrapped lozenges 50 may be sold together in bags of the same flavor or a single bag may have different flavors. The flavor may be indicated on the bag or on the individual wrappers 53 themselves.

Conventional lozenges have a specific medicating agent or a certain active ingredient that performs the soothing, lubricating, healing, or comforting action. For example, conventional lozenges may include a pharmaceutically active agent. The term “pharmaceutically active agent” refers to any agent or compound that is usually used to treat a certain condition or disease (may or may not be approved by the U.S. Food and Drug Administration, European Medicines Agency, or any successor entity thereof, for the oral treatment of a condition or disease). Examples of pharmaceutically active agents include, but are not limited to, analgesics, anti-inflammatory agents, antipyretics, antihistamines, antibiotics (e.g., antibacterial, antiviral, and antifungal agents), antidepressants, antidiabetic agents, antispasmodics, appetite suppressants, bronchodilators, cardiovascular treating agents (e.g., statins), central nervous system treating agents, cough suppressants, decongestants, diuretics, expectorants, gastrointestinal treating agents, anesthetics, mucolytics, muscle relaxants, osteoporosis treating agents, stimulants, and sedatives.

While lozenges 50 may be used to treat a variety of conditions and diseases, one of the most prevalent and widespread uses of lozenges is to soothe and treat a sore throat. Sore throats are generally caused by a bacterial or viral presence in the throat of a user. While there are many different manufactured agents and compounds that have been formulated to fight bacteria and viruses, silver has been shown to have germicidal properties. Silver has been employed as a germicide and antibiotic for a number of years and was used by civilization before modern antibiotics were developed. For example, in previous centuries users would shave silver particles into their drinking water, or submerge whole silver pieces in the drinking water, for the purpose of ingesting the silver by drinking. Accordingly, the present disclosure relates specifically to implementing silver nanoparticles in throat lozenge compositions. Additional details relating to silver nanoparticles are included below with reference to FIG. 2A.

FIG. 2A is a schematic block diagram of a solid lozenge composition 100, according to one embodiment. According to the depicted embodiment, a solid lozenge composition may include an inactive carrier medium 102, a flavoring component 104, and silver nanoparticles 106, among others.

The inactive carrier medium 102 is generally the most prevalent constituent in the lozenge 50. The inactive carrier medium 102 generally doesn't have any healing, soothing, or medicating properties but instead constitutes the main ingredient into which the other ingredients are mixed/dispersed. According to one embodiment, the inactive carrier medium 102 may be an evaporated cane juice, such as Organic Evaporated Cane Juice made by Florida Crystals of West Palm Beach, Fla. The inactive carrier medium 102 may be selected according to its solubility properties. Since the carrier medium 102, at least according to one embodiment, is the main constituent in the lozenge composition 100 (e.g., the inactive carrier medium may comprise more than 50 volume percent of the solid lozenge composition 100), the dissolution rate of the lozenge 50 in the mouth of user is largely dependent on the solubility of the carrier medium 102. According to another embodiment, the carrier medium 102 is selected according to its taste, flavor, color, consistency, nutritional value, etc.

The lozenge composition 100 also includes at least one flavoring component 104. In one embodiment, the carrier medium 102 may have a sufficiently strong flavor that a separate, independent flavoring component 104 is not required. However, in most implementations a separate flavoring component 104 will be included in the lozenge composition 100. The flavoring component 104 may be a sweetener or an aromatic compound. In one embodiment, the flavoring component 104 is an essential oil, such as peppermint oil.

Examples of sweeteners include, but are not limited to, synthetic or natural sugars; artificial sweeteners such as saccharin, sodium saccharin, aspartame, acesulfame, thaumatin, glycyrrhizin, sucralose, dihydrochalcone, alitame, miraculin, monellin, and stevside; sugar alcohols such as sorbitol, mannitol, glycerol, lactitol, maltitol, and xylitol; sugars extracted from sugar cane and sugar beet (sucrose), dextrose (also called glucose), fructose (also called laevulose), and lactose (also called milk sugar); isomalt, salts thereof, and mixtures thereof.

Examples of flavors and aromatics include, but are not limited to, essential oils including distillations, solvent extractions, or cold expressions of chopped flowers, leaves, peel or pulped whole fruit comprising mixtures of alcohols, esters, aldehydes and lactones; essences including either diluted solutions of essential oils, or mixtures of synthetic chemicals blended to match the natural flavor of the fruit (e.g., strawberry, raspberry and black currant); artificial and natural flavors of brews and liquors, e.g., cognac, whisky, rum, gin, sherry, port, and wine; tobacco, coffee, tea, cocoa, and mint; fruit juices including expelled juice from washed, scrubbed fruits such as lemon, orange, and lime; spear mint, pepper mint, wintergreen, cinnamon, cacoe/cocoa, vanilla, liquorice, menthol, eucalyptus, aniseeds nuts (e.g., peanuts, coconuts, hazelnuts, chestnuts, walnuts, colanuts), almonds, raisins; and powder, flour, or vegetable material parts including tobacco plant parts, e.g., genus Nicotiana, in amounts not contributing significantly to the level of nicotine, and ginger.

As described above, lozenges 50 may contain various active ingredients and/or medications. Although conventional lozenges 50 may have various pharmaceutical compounds or manufactured chemicals in the form of antibiotics or antibacterial agents, the present disclosure relates to lozenges that have elemental silver. While various colloidal or ionic silver solutions may be implemented as the silver nanoparticles 106 in the present disclosure, it is anticipated that the particular form of silver nanoparticles described below will be especially useful in the lozenge composition 100 of the present disclosure.

There may be many reasons why orally administering silver would enhance an individual's health. It is anticipated that silver operates to inhibit the growth of bacteria, viruses, and other unwanted organisms, as well as eradicating such existing bacteria, viruses, and other organisms. It is also possible that a solution of silver can have an anti-inflammatory effect, sufficient to reduce symptoms of asthma. Silver in solution might also act in a similar fashion to a homeopathic remedy. These are just a few of the possible reasons why silver in solution, such as colloidal silver, is effective at enhancing health.

Attempts have been made in the prior art to produce silver-based solutions, including colloidal silver, some of which have been more successful than others. Many of the presently available silver-based products, however, are unstable and lose the silver to precipitation (i.e., silver ‘falls out” of solution). A true colloid operates to maintain the colloidal particles in suspension over a period of several years, and perhaps indefinitely. However, many of the conventional silver products fail to maintain the silver particles in suspension, either because the silver solution is not a true colloid or because it is otherwise unstable. When the suspension of the silver particles fails, the particles fall to the bottom of the solution, thereby reducing the solution's concentration of silver and rendering it less effective. This is especially true when dealing with lozenges. If the silver nanoparticles are not evenly dispersed throughout the lozenge 50, the bacterial or viral infection source in the throat of a user will not receive constant and uniform contact with the silver nanoparticles and the lozenge 50 will thus have a decreased effectiveness in treating the user's ailment.

Several U.S. patents describe various ways of making a silver-based solution. However, these patents fail to teach or suggest a process by which stable, colloidal silver may be produced in larger batch quantities and at increased rates of production. Even further, these reference fail to show how a stable colloidal silver can be implemented in conjunction with an inactive carrier medium 102 and a flavoring component 104 to make solid lozenges 50 that can effectively treat bacteria and viruses found in the throat of a user.

In one embodiment, the stable silver nanoparticles are made by placing a silver electrode in contact with a quantity of high purity water, conveying electrical current through the silver electrode to separate particles of silver from the silver electrode and then agitating the water to disperse the silver particles into a more uniform concentration within the water such that a higher quantity of suspended silver particles can be produced per batch. Thus, according to one embodiment, silver particles produced in this manner (i.e., via electrolysis and agitation) are referred to as ‘silver nanoparticles’ in the present disclosure. These particles, according to one embodiment, may be bonded to the water molecules, thus resulting in a substantially stable mixture/suspension of silver nanoparticles. In one embodiment, the silver nanoparticles are produced directly in the inactive carrier medium (i.e., silver bonded to inactive carrier medium molecules via electrolysis, etc). In another embodiment, the silver nanoparticles are produced in an aqueous solution and then upon combining the solution with the inactive carrier medium and subsequent heating/mixing, the water may boil off, leaving behind a substantially uniformly dispersed suspension of silver nanoparticles in the inactive carrier medium.

The term ‘silver nanoparticles’ is used because the silver particles produced in the above described manner are small enough to provide substantial surface area, thus increasing the rate and frequency of surface reactions with the bacteria/virus, but large enough to prevent a loss of stability caused by particles that are too small. Also, the silver nanoparticles are essentially colorless while other colloidal silver preparations (particularly with larger particle sizes) usually show colors. Digital analysis of the silver nanoparticles produced according to the above description showed an average particle diameter of 0.0106 micrometers with a range of 0.005 micrometer to 0.0851 micrometers. However, size distribution analysis shows that more than 95% of the particles were between about 0.005 micrometers and about 0.015 micrometers in diameter. At these sizes, the silver nanoparticles 106 are well suited to remain in solution as a colloid suspension, thus increasing the effectiveness of the lozenge 50.

According to one embodiment, the silver nanoparticle 106 used in the lozenge composition 100 is an aqueous solution containing deionized water and suspended silver particles. According to one embodiment, the concentration of the silver particles in the precursor silver solution may be between about 5 and 50 parts per million (ppm). The silver nanoparticles may, in precursor form (before combining with the other constituents of the lozenge 50) be ASAP Silver Solutions made by American Biotech Labs of Alpine, Utah.

FIG. 2B is a schematic block diagram of the solid lozenge composition 100, according to another embodiment. The lozenge composition 100 may include an inactive carrier medium 102, a flavoring component 104, silver nanoparticle 106, a consistency modifier 208, and a dispersing medium 210. The inactive carrier medium 102, the flavoring component 104, and the silver nanoparticle 106 are described above with reference to FIG. 2A. The consistency modifier 208 and the dispersing medium 210 are described below.

The consistency modifier 208 may be any constituent that alters the consistency, uniformity, viscosity, smoothness, thickness, hardness rating, brittle nature, or other physical property of the composition 100. The consistency modifier 208 may also have flavor, taste, smell, and color characteristics that contribute to the overall function of the lozenge 50. According to one embodiment, the consistency modifier 208 is honey or nectar from a fruit, such as agave nectar. In such implementations, the consistency modifier 208 (e.g., honey) not only contributes to the consistency and “smoothness” of the lozenge, but also contributes greatly to the overall taste and flavor of the lozenge 50. Thus, in one embodiment the consistency modifier 208 may actually be a second flavoring component 104 and vice-versa. In other words, distinguishing one constituent as either a flavoring component 104 or as a consistency modifier 208 is not an exclusive identifier and labeling certain constituents as either a flavoring component 104 or a consistency modifier 208 should not be construed to limit the scope of the present disclosure. Additionally, the consistency modifier 208 and/or the flavoring component 104 may affect the overall solubility and ‘dispersability’ of the lozenge 50, therefore the consistency modifier 208 and the flavoring component 208 may have attributes that are comparable to the inactive carrier medium 102.

As briefly described above, the silver nanoparticles 106 may be combined with the other constituents of the lozenge composition 100 in the form of a solution. The solvent in which the silver particles are suspended is referred to as the dispersing medium 210. Thus, the dispersing medium 210 may also be present in the lozenge composition 100. In one embodiment, the dispersing medium 210 is water. As described below, in certain implementations the dispersing medium 210 may be boiled off during the procedure of making the lozenges 50. However, it is possible that the dispersing medium 210 may remain in the lozenge composition 100, thus contributing to the consistency, taste, solubility, etc. of the lozenge 50.

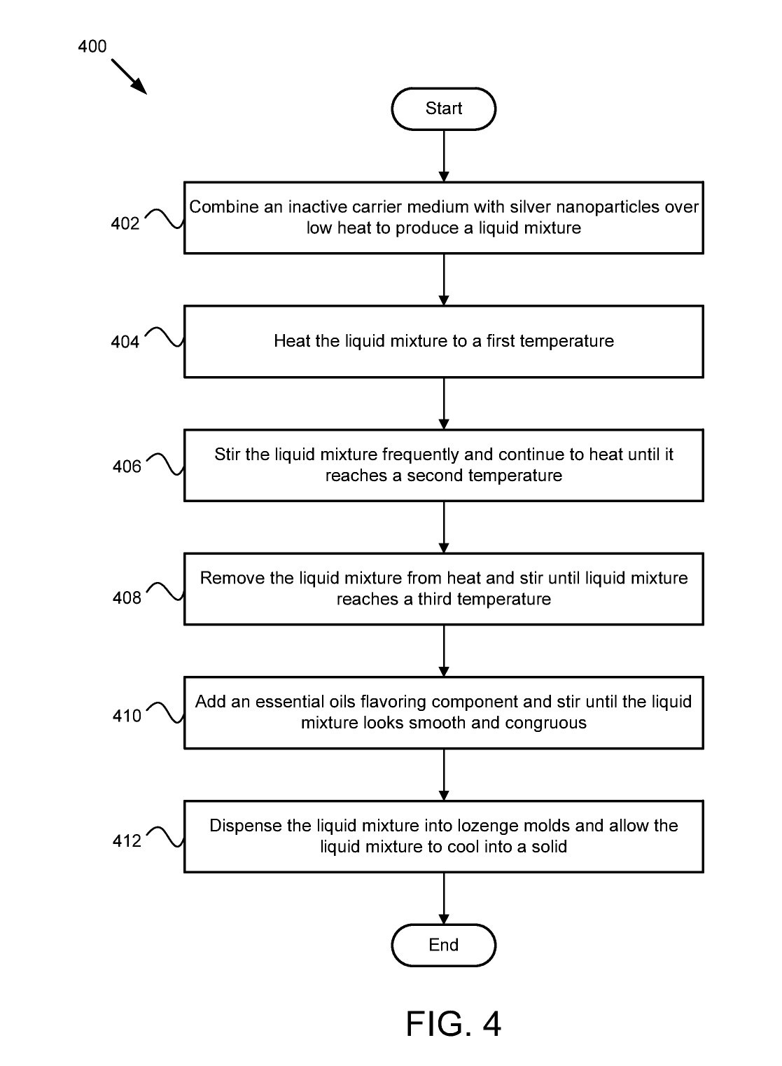

FIG. 3A depicts a user orally taking a solid lozenge 50, according to one embodiment. According to one embodiment, the lozenge 50 of the present disclosure may contain evaporated cane juice as the inactive carrier medium 102 at a volume fraction of greater than 50% of the total volume of the lozenge composition 100. The lozenge 50 may also contain honey as the consistency modifier 208 at a volume fraction of about 20%, an aqueous silver solution (5-50 ppm silver) as the silver nanoparticle 106/dispersing medium 210 at a volume fraction of about 20%, and a small volume of essential oil (e.g., peppermint) as the flavoring component 104. As described below with reference to FIG. 4, a bulk batch of lozenge composition 100 may be divided into individual lozenges 50 for oral ingestion.

In one embodiment, an aqueous silver solution, made according to the method described above where silver nanoparticles are bonded to water molecules, is implemented as the silver nanoparticle 106/dispersing medium 210 and contains 30 ppm silver. In one embodiment, the final concentration of silver in the final lozenge composition is between about 1 ppm and 25 ppm. In another embodiment, the final concentration of silver in the final lozenge composition is between about 2 ppm and 10 ppm. In yet another embodiment, the final concentration of silver in the final lozenge composition is about 4.25 ppm. In other words, 19 micrograms per 4.5 gram lozenge.

FIG. 3B depicts a user orally taking a solid lozenge 50, according to another embodiment. Depending on the overall solubility of the lozenge 50 and the size of the individual lozenges 50, it may take anywhere from a few minutes to 10's of minutes for the lozenge to completely dissolve in the mouth of a user. Depending on the source of discomfort within the throat 60 (i.e., bacterial, viral), the silver nanoparticles 106 may need to be in contact with the throat for a certain period of time in order to effectively treat, kill, eliminate, or neutralize the bacterial/viral infection. In one embodiment, the lozenges 50 may be designed to last for at least 6 minutes before completely dissolving, thus providing the user with at least 6 minutes of silver contacting the bacterial or viral source in the throat 60.

FIG. 4 is a schematic flow chart diagram of a method 400 for making a solid lozenge 50, according to one embodiment. The method 400 includes combining 402 an inactive carrier medium 102 with silver nanoparticles 106 over low heat to produce a liquid mixture. According to one embodiment, the concentration of silver nanoparticles in the mixture is between about 0.1 micrograms per teaspoon and 5 micrograms per teaspoon. The method 400 further includes heating 404 the liquid mixture to a first temperature before stirring 406 the liquid mixture frequently while heating the liquid mixture to a second temperature. In one embodiment, the first temperature is 250 degrees Fahrenheit and the second temperature 295 degrees Fahrenheit. Still further, the method 400 includes removing 408 the liquid mixture from heat while continuing to stir until the temperature of the liquid mixture falls back down to a third temperature, which may be about 280 degrees Fahrenheit. An essential oils flavoring component 104, according to one embodiment, is then added 410 to the liquid mixture and the liquid mixture is stirred until it looks smooth and congruous. Finally, the method 400 includes dispensing 412 the liquid mixture into lozenge molds and allowing the liquid mixture to cool into a solid.

During the heating 404 step of the method 400, the dispersing medium 210 may be water, which as a boiling point of 212 degrees Fahrenheit. In such an embodiment, the dispersing medium 210 may boil off before the temperature of the liquid mixture continues to rise. In situations where the dispersing medium 210 boils off, the final lozenge composition 100 will be affected by losing a certain volume of water. A manufacturer will need to keep this in mind when targeting a final lozenge composition 100 that has a certain concentration of silver nanoparticles 106 (i.e., the active ingredient).

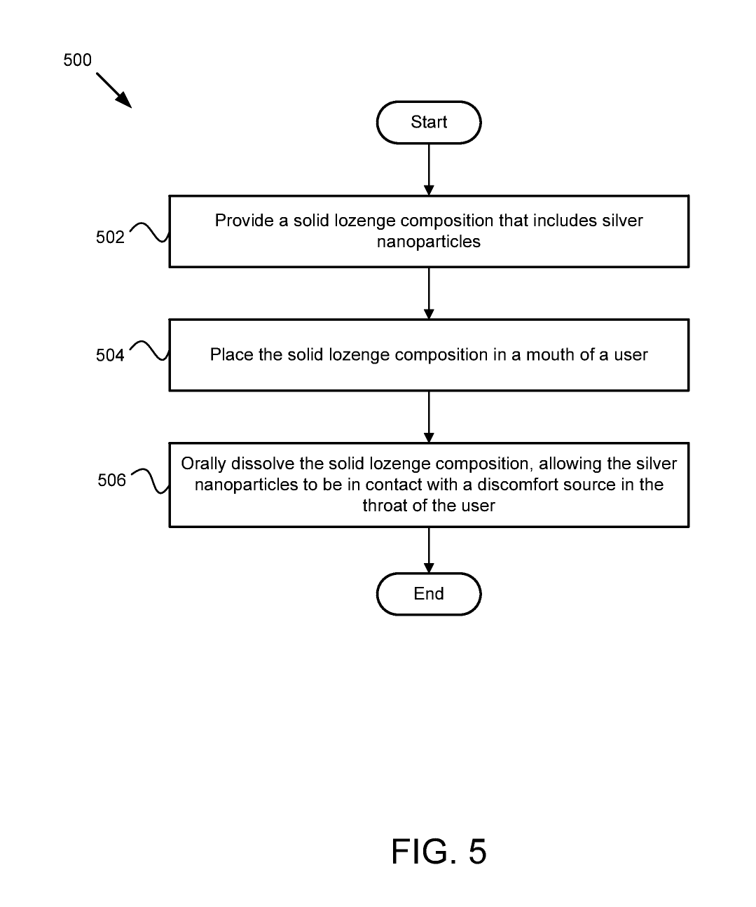

FIG. 5 is a schematic flow chart diagram of one embodiment of a method 500 for using solid lozenges containing silver nanoparticles. The method 500 includes providing 502 a solid lozenge composition for oral dissolution. The solid lozenge composition includes an inactive carrier medium that is a solid at room temperature, at least one flavoring component, and silver nanoparticles, wherein the concentration of silver nanoparticles is between about 0.1 micrograms per teaspoon and 5 micrograms per teaspoon. The method 500 further includes placing 504 the solid lozenge composition in a mouth of a user and then orally dissolving 506 the solid lozenge composition for a time period of at least 2 minutes, during which time period the silver nanoparticles are in substantially constant and repeated contact with a discomfort source in the throat of the user. According to one implementation, the time period is at least 6 minutes.

Reference throughout this specification to features, advantages, or similar language does not imply that all of the features and advantages that may be realized with the subject matter of the present disclosure should be or are in any single embodiment of the subject matter. Rather, language referring to the features and advantages is understood to mean that a specific feature, advantage, or characteristic described in connection with an embodiment is included in at least one embodiment of the subject matter of the present disclosure. Thus, discussion of the features and advantages, and similar language, throughout this specification may, but do not necessarily, refer to the same embodiment.

Furthermore, the described features, structures, advantages, and/or characteristics of the subject matter of the present disclosure may be combined in any suitable manner in one or more embodiments and/or implementations. In the following description, numerous specific details are provided to impart a thorough understanding of embodiments of the subject matter of the present disclosure. One skilled in the relevant art will recognize that the subject matter of the present disclosure may be practiced without one or more of the specific features, details, components, materials, and/or methods of a particular embodiment or implementation. In other instances, additional features and advantages may be recognized in certain embodiments and/or implementations that may not be present in all embodiments or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims, or may be learned by the practice of the subject matter as set forth hereinafter.

Similarly, reference throughout this specification to “one embodiment,” “an embodiment,” or similar language means that a particular feature, structure, or characteristic described in connection with the embodiment is included in at least one embodiment of the subject matter of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the subject matter of the present disclosure, however, absent an express correlation to indicate otherwise, an implementation may be associated with one or more embodiments.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. A solid lozenge composition for oral dissolution comprising:
an inactive carrier medium that is a solid at room temperature;
at least one flavoring component; and
silver nanoparticles, wherein the concentration of silver nanoparticles in the solid lozenge composition is between about 0.1 ppm and 25 ppm.

2. The solid lozenge composition of claim 1, further comprising a silver dispersing medium that, independent from the inactive carrier medium, is a liquid at room temperature.

3. The solid lozenge composition of claim 1, wherein the solubility of the solid lozenge composition in a mouth of a user and the size of an individual lozenge are such that it takes at least 6 minutes to completely dissolve each individual lozenge.

4. The solid lozenge composition of claim 1, wherein the inactive carrier medium comprises more than or equal to 50 volume percent of the solid lozenge composition.

5. The solid lozenge composition of claim 1, wherein the silver nanoparticles are produced via electrolysis.

6. The solid lozenge composition of claim 1, wherein the inactive carrier medium is evaporated cane juice.

7. The solid lozenge composition of claim 1, further comprising a consistency modifier.

8. The solid lozenge composition of claim 7, wherein the consistency modifier comprises honey.

9. The solid lozenge composition of claim 1, wherein the flavoring component comprises an essential oil.

10. The solid lozenge composition of claim 1, wherein the concentration of silver nanoparticles in the solid lozenge composition is between about 2 ppm and 10 ppm.

11. The solid lozenge composition of claim 1, wherein the concentration of silver nanoparticles in the solid lozenge composition is about 4.25 ppm.

12. The solid lozenge composition of claim 1, wherein the concentration of silver nanoparticles in an individual lozenge is about 19 micrograms.

13. A method for using solid lozenges containing silver nanoparticles, the method comprising:
providing a solid lozenge composition for oral dissolution, the solid lozenge composition comprising an inactive carrier medium that is a solid at room temperature, at least one flavoring component, and silver nanoparticles, wherein the concentration of silver nanoparticles is between about 0.1 ppm and 25 ppm;
placing the solid lozenge composition in a mouth of a user; and
after placing the solid lozenge composition in the mouth of the user, orally dissolving the solid lozenge composition for a time period of at least 2 minutes, during which time period the silver nanoparticles are in substantially constant and repeated contact with a discomfort source in the throat of the user.

14. The method of claim 13, wherein the time period is at least 6 minutes.

15. A method for making solid lozenges containing silver nanoparticles, the method comprising:
combining an inactive carrier medium with silver nanoparticles over low heat to produce a liquid mixture, wherein the concentration of silver nanoparticles in the mixture is between about 0.1 ppm and 25 ppm;
heating the liquid mixture to a first temperature;
after heating the liquid mixture to the first temperature, stifling the liquid mixture frequently and heating the liquid mixture to a second temperature;
after heating the liquid mixture to the second temperature, removing the liquid mixture from heat and continue stifling until the temperature of the liquid mixture falls to a third temperature;
after the temperature of the liquid mixture falls to the third temperature, adding at least one essential oils flavoring component and continue stifling until the liquid mixture looks smooth and congruous; and
dispensing the liquid mixture into lozenge molds and allowing the liquid mixture to cool into a solid.

16. The method of claim 15, further comprising initially combining a consistency modifier with the inactive carrier medium and the silver nanoparticles to produce the liquid mixture.

17. The method of claim 15, wherein the second temperature is higher than the first temperature and the third temperature is between the first and the second temperatures.

18. The method of claim 15, wherein the first temperature is 250 degrees Fahrenheit.

19. The method of claim 18, wherein the second temperature is 295 degrees Fahrenheit.

20. The method of claim 19, wherein the third temperatures is 280 degrees Fahrenheit.
